(19) United States
(12) Patent Application Publication
(10) Pub. No.: US 2009/0143673 A1
(43) Pub. Date: Jun. 4, 2009

(54) TRANSIT TIME ULTRASONIC FLOW MEASUREMENT

(75) Inventors: Cornelis J. Drost, Ithaca, NY (US); Yuri M. Shkarlet, Pompano Beach, FL (US); Bruce McKee, Ithaca, NY (US); Kevin Lutkins, Union Springs, NY (US)

Correspondence Address: Stephen B. Salai, Esq.;Harter Secrest & Emery LLP, 1600 Bausch & Lomb Place, Rochester, NY, 14604-2711, US

(73) Assignee: Transonic Systems Inc., Ithaca, NY (US)

(21) Appl. No.: 12/325,096

(22) Filed: Nov. 28, 2008

Related U.S. Application Data
(60) Provisional application No. 61/004,858, filed on Nov. 30, 2007.

(51) Int. Cl. A61B 8/00; G01F 1/00; G01F 1/66

(52) U.S. Cl. 600/437; 73/861; 73/861.18; 73/861.28

(57) ABSTRACT

A transcutaneous energy transfer system with subcutaneous non coupled coils is used to transmit power and signals to an implanted biological support device or sensor, such as a flow sensor for measuring relatively low flow rates, such as hydrocephalic shunt flow. The flow sensor is configured to convert a shear wave generated by a transducer to a longitudinal wave at the interface of a signal pathway and the flow, wherein the longitudinal wave travels parallel to the flow and exits a flow channel to convert to a shear wave which intersects a second transducer. The transcutaneous energy transfer employs a pair of inductive coupling coils, wherein the coils are disposed in zero coupling orientation which can include a perpendicular orientation of corresponding coil axes.

### CROSS-REFERENCE TO RELATED APPLICATIONS

The present application claims the benefit of U.S. provisional patent application No. 61/004,858, filed Nov. 30, 2007.

### STATEMENT REGARDING FEDERALLY SPONSORED RESEARCH OR DEVELOPMENT

This invention was made with government support under Research supported under Phase-I SBIR (Small Business Innovative Research) Grant #1R43 NS049680-01A1 from the National Institute of Neurological Disorders and Stroke of the National Institutes of Health. The government has certain rights in the invention.

### REFERENCE TO A “SEQUENCE LISTING”

Not applicable.

### BACKGROUND OF THE INVENTION

1. Field of the Invention

The present invention relates to a transcutaneous energy transfer system as well as a transit time ultrasonic sensor, and more particularly to coupled inductive coils and an ultrasonic flow sensor that can be employed in relatively low flow systems, such as hydrocephalic shunt flow measurement.

2. Description of Related Art

Ventricles of the brain contain cerebrospinal fluid (CSF) which cushions the brain against shock and provides a means for nutrient and waste transport in the brain. CSF is constantly being secreted and absorbed by the body, usually in equilibrium. However, if blockages exist in the circulation pathways of CSF, the CSF can't be reabsorbed by the body at the proper rate.

This imbalance can create a condition known as hydrocephalus: a condition marked by an excessive accumulation of fluid in subarachnoid space, including the cerebral ventricles. Hydrocephalus is a condition characterized by abnormal flow, absorption or formation of CSF which may subsequently increase the volume and/or pressure of the intracranial cavity. If left untreated, the increased intracranial pressure can lead to neurological damage and may result in death.

In addition, it has been found that in some persons with adult-onset dementia of the Alzheimer's type there is dysfunction of the cerebrospinal fluid resorptive mechanism, leading to the retention in the cerebrospinal fluid of substances which result in the histologic lesions associated with adult-onset dementia of the Alzheimer's type, or which are neurotoxic, or both.

Therefore, in both the treatment of hydrocephalus and adult-onset dementia of the Alzheimer's type, there is a need to remove excess CSF.

A CSF shunt is a common treatment for hydrocephalus patients. A standard shunt consists of the proximal (upstream) catheter, a valve and a distal (downstream or discharge) catheter. The excess CSF is typically drained from the ventricles or other subarachnoid locations to a suitable cavity, most often the peritoneum or the right atrium of the heart. The shunt thereby relieves pressure from the CSF acting on the brain.

Typically, a physician selects a pressure-flow relationship for the CSF of the patient. Current shunts allow the physician to select pressure settings, but in situ CSF flow itself—the very purpose of placing the shunt—is unknown and must be derived from clinical signs.

The existence of shunt dysfunction further complicates treatment. Hydrocephalic shunt dysfunction diagnosis is one of the most complicated and time consuming aspects of treating hydrocephalic infants and young children. Hydrocephalus can require lifelong treatment with implanted shunt systems that drain excess CSF. However, pediatric hydrocephalic patients have a high risk of shunt dysfunction. It has been estimated that 25% to 40% of the shunts fail in the first year of implantation.

Diagnosis of shunt dysfunction is particularly challenging with preverbal children as such children cannot describe critical symptoms such as headaches. Diagnosis must instead rely on observable symptoms that often resemble those of common childhood illnesses. Unfortunately, the absence of an objective shunt dysfunction diagnostic method leads to delayed treatment or unnecessary intervention.

Neurosurgeons currently diagnose shunt dysfunctions using a combination of neurological studies, shunt pumping, shunt tapping, nuclear or x-ray contrast studies (“shuntograms”), and CT (computed tomography) scans. However, none of these methods actually measure the volumetric flow in the shunt.

Shunt pumping involves depressing a shunt reservoir to see if the reservoir empties and then refills. This method is often inconclusive since the reservoir is typically made of relatively soft materials that can be easily distorted and depressed without creating any fluid motion.

More definitive results can be obtained by shunt tapping and nuclear flow studies. Shunt tapping involves inserting a needle into the ventricular catheter reservoir to directly access the CSF. Doctors detect proximal blockage by measuring CSF drainage and by observing manometer fluctuations. However, this technique risks shunt infection; does not diagnose the other parts of the shunt system; can be inconclusive in patients with small ventricles and low pressure; and results can be complicated by physiological disturbances caused by a child expressing severe pain and distress.

While shunt tapping can detect proximal occlusion, nuclear flow or X-ray contrast studies can provide information on distal occlusions. In nuclear flow studies, radioactive isotopes are injected into the shunt, and a gamma camera monitors flow of the isotope through the shunt. In X-ray contrast studies, an X-ray contrast agent is injected in the shunt and flow of the agent is monitored via periodic patient x-rays. While these tests can determine shunt patency and shunt flow, this may not be a preferred procedure in many instances since it requires involved expensive studies that will provide readings only while the patient is restrained to a fixed position rather than the dynamic flow picture under different patient conditions such as sitting up and lying down.

Head CT scans can directly visualize the ventricles and show the proximal shunt catheter position. These scans are readily available in most hospitals, and the CT scan can define CSF volumes and changes in ventricular volume, but the CT scan cannot measure flow. Also, stable ventricular volume does not indicate shunt patency, since a patient with very small ventricles can show no change in flow even with an occluded shunt and high intra-cranial pressure. Finally, each scan exposes the patient to additional radiation, a well known health hazard.

Doppler ultrasound relies on backscatter of ultrasonic waves on red blood cells. Since no such cells are present in the CSF, a Doppler sensor is not commercially available.

Therefore, there is a need to measure CSF shunt flow to assess shunt patency and under drainage or over drainage. There is a further need to non-invasively measure shunt CSF flow. The need to measure CSF flow can reduce tests, improve clinical outcomes, avoid unnecessary shunt replacement surgeries, and reduce patient/parental stress.

The need also exists for a non-invasive, relatively inexpensive method to measure CSF shunt flow that can be employed at more hospitals and emergency rooms. A need further exists for the quantification of the actual volume flow (ml/hr) carried by the CSF shunt. A need also exists for providing transcutaneous energy transfer to a subcutaneous biological device.

### BRIEF SUMMARY OF THE INVENTION

The present disclosure provides an implantable sensor assembly having an implantable housing; a first internal coil retained within the housing, the first internal coil having a first coil axis; a second internal coil retained within the housing, the second internal coil having a second coil axis; the second coil axis being nonparallel to the first coil axis; and a sensor connected to the housing and electrically coupled to at least one of the first internal coil and the second internal coil.

The sensor can include a first transducer and a second transducer, wherein the first transducer is electrically connected to the first internal coil and the second transducer is electrically connected to the second internal coil. It is contemplated that the sensor can be an ultrasonic flow sensor. The ultrasonic flow sensor can define a flow channel with integral signal pathways defining a portion of the flow channel.

A method is provided for the transcutaneous transfer of a transmitting and receiving signal between an external flowmeter device and the internal flow sensor transducers, by subcutaneously locating a first internal coil and a second internal coil, the first internal coil aligned along a first axis and the second internal coil aligned along a nonparallel second axis; and inductively coupling a first external coil with the first internal coil, and a second external coil with the second internal coil, the first external coil and the first internal coil aligned parallel to the first axis and the second external coil and the second internal coil aligned parallel to the second axis. The internal flow sensor transducers connect to the first internal coil and the second internal coil, and are thus electronically connected to the external flowmeter. The first and second coil axes are generally located such that the coils do not exhibit mutual inductance, so that a signal or power passing through one of the coils does not induce a corresponding signal or power in the remaining coil.

A method is also provided wherein power and signals are transcutaneously transferred between an external electronic apparatus and an implanted biologic device, by subcutaneously locating a biologic device, a first internal coil and a second internal coil, the first internal coil aligned along a first axis and the second internal coil aligned along a nonparallel second axis; inductively coupling a first external coil with the first internal coil, and a second external coil with the second internal coil; energizing the biologic device from one of the first internal coil and the second internal coil, and transferring signal energy from the biological device to the other internal coil back to the external electronic apparatus. With respect to the internal and external coils, the method further includes aligning the first and second axes of the internal and external coils so as to connect the internal sensor inductively to the external electronics. The first and the second axes are generally selected to have a relationship such that the energy transfer via a coil aligned along one axis will minimally interfere with the signal transfer via a coil aligned along the other axis.

A transcutaneous energy transfer assembly is also provided wherein an external housing retains (i) a first external coil having a first coil axis and (ii) a second external coil having a second coil axis; the first coil axis being nonparallel to the second coil axis; and wherein an implantable housing retains (i) a first internal coil having a first internal coil axis and (ii) a second internal coil having a second internal coil axis; the first internal coil axis being nonparallel to the second internal coil axis.

A sensor can be at least partially retained within the implantable housing, wherein the sensor and/or its interfacing electronics is electrically connected to the first internal coil and the second internal coil. It is further contemplated that an orientation between the first external coil axis and the second external coil axis is the same as the orientation between the first internal coil axis and the second internal coil axis.

The present flow sensor also includes a housing formed of a first material, the housing defining a flow channel having a linear section bounded by a first bend and a second bend, the first material of the housing forming a first signal pathway adjacent the first bend and a second signal pathway adjacent the second bend; a first transducer adjacent the first signal pathway; and a second transducer adjacent the second signal pathway.

The transducers can be directly connected or acoustically coupled to the respective signal pathway in the housing. The transducers can produce one of a longitudinal or a shear wave to pass along the signal pathways, wherein the signal pathway is selected to refract the wave into a fluid flow in the flow channel to propagate along the measurement channel. If the transducers are shear-wave transducers, the pathway is designed such that a shear wave converts to a longitudinal wave at one of the boundaries within the signal pathways, typically the boundary between the housing and the flow channel.

### BRIEF DESCRIPTION OF THE SEVERAL VIEWS OF THE DRAWING(S)

FIG. 1 is a schematic representation of a patient having a hydrocephalic shunt in cooperation with a long term implanted flow sensor and external flow meter and coupling coils.

FIG. 2 is a schematic representation of a first configuration of a flow channel in a flow sensor.

FIG. 3 is a schematic representation of a second configuration of a flow channel in the flow sensor.

FIG. 4 is a perspective view of components forming the implantable housing.

FIG. 5 is a top plan view of the assembled housing of FIG. 2.

FIG. 6 is a bottom plan view of the assembled housing of FIG. 2.

FIG. 7 is an end view of the assembled housing of FIG. 2.

FIG. 8 is a second end view of the assembled housing of FIG. 2.

FIG. 9 is a plot of refraction angles versus angle of incidence for a selected material of the housing and signal pathways.

FIG. 10 is a schematic view of an alternative configuration of a flow channel.

FIG. 11 is a perspective view of the transcutaneous energy transfer system in conjunction with an implantable sensor.

FIG. 12 is a perspective view of an implantable housing with internal coupling coils wound about a portion of the housing, prior to encapsulation.

FIG. 13 is an algorithm for providing a zero offset compensation in response to dynamic changes in separation between implanted internal coils and external coils resulting from patient motion.

FIG. 14 is a graph of data from a sheep in-vivo study, in which flow was stopped to assess the effects of sheep motion on flow zero offset, wherein the graph shows how changes in zero offset track changes in the received signal voltage amplitude.

FIG. 15 is a graph showing correlation of changes in coil separation with flow zero offset changes from in-vitro studies.

FIG. 16 is a graph showing correlation of actual flow to a zero offset compensated measure of flow.

FIG. 17 is a graph showing zero offset drift correction based on the amplitude of the received signal.

FIG. 18 is a flow chart of the functioning of the implanted flow sensor and the transcutaneous energy transfer.

FIG. 19 is a schematic representation of the flow sensors employed in an extracorporeal system.

FIG. 20 is a perspective view of the extracorporeal flow sensor.

FIG. 21 is a schematic representation of the transcutaneous energy transfer to one of an implanted biologic support device and sensor, with the energy transfer being spaced subcutaneously remote from the one of the biologic support device and sensor.

### DETAILED DESCRIPTION OF THE INVENTION

The present disclosure provides a transcutaneous energy transfer (TET) system, wherein the TET system can be employed to transfer power and signal energy between any of a variety of subcutaneous devices, which include implanted biological support devices and sensors. The biological support devices include pumps for introducing medicines, tracers or indicators, dispensers, heaters, coolers and even electrical stimulators. The implanted sensors include flow, pressure, ECG, EEG, EMG, PH, and blood properties. For purposes of description, the present description is set forth in terms of an implanted ultrasonic transit time flow sensor. Further, although the present flow measurements are set forth in terms of low flow shunt measurements and particularly hydrocephalic shunts, the invention is not limited to such specific systems. The flow sensor and the transcutaneous energy and signal transfer are not so limited and can be employed in any of a variety of applications, such as biomedical device applications.

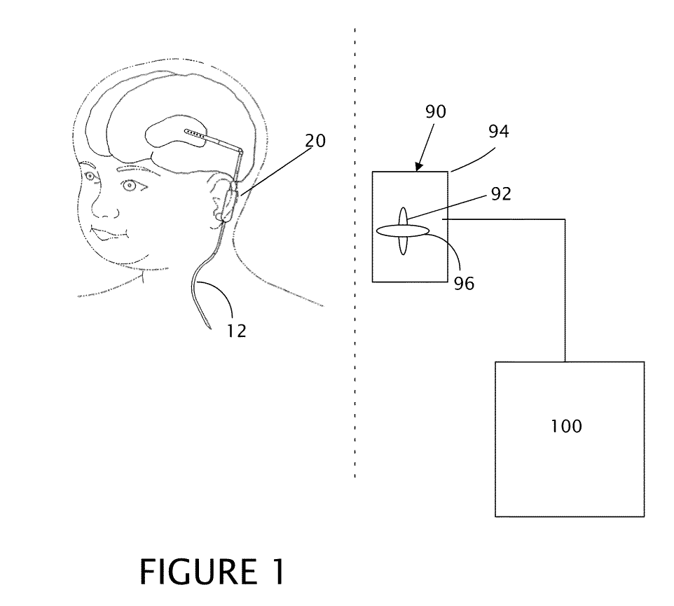

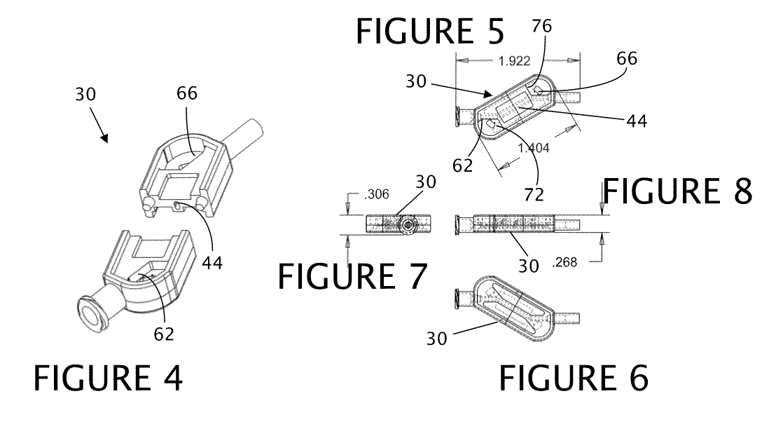

Referring to FIGS. 1-12, a hydrocephalic shunt 12 is shown operably connected to a long term implantable sensor 20 in cooperation with external or readout coupling coils 90 and a flowmeter 100.

Referring FIGS. 1, 4 and 12, the implantable sensor 20 includes an implantable housing 30, wherein internal coupling coils 80 and a flow sensor 22 are encapsulated with the housing. That is, the housing 30 can retain and encapsulate the internal coupling coils 80 or alternatively the internal coupling coils can be commonly encapsulated within the housing. In selected configurations, the housing 30 can also retain at least one of an operating circuit and signal condition components.

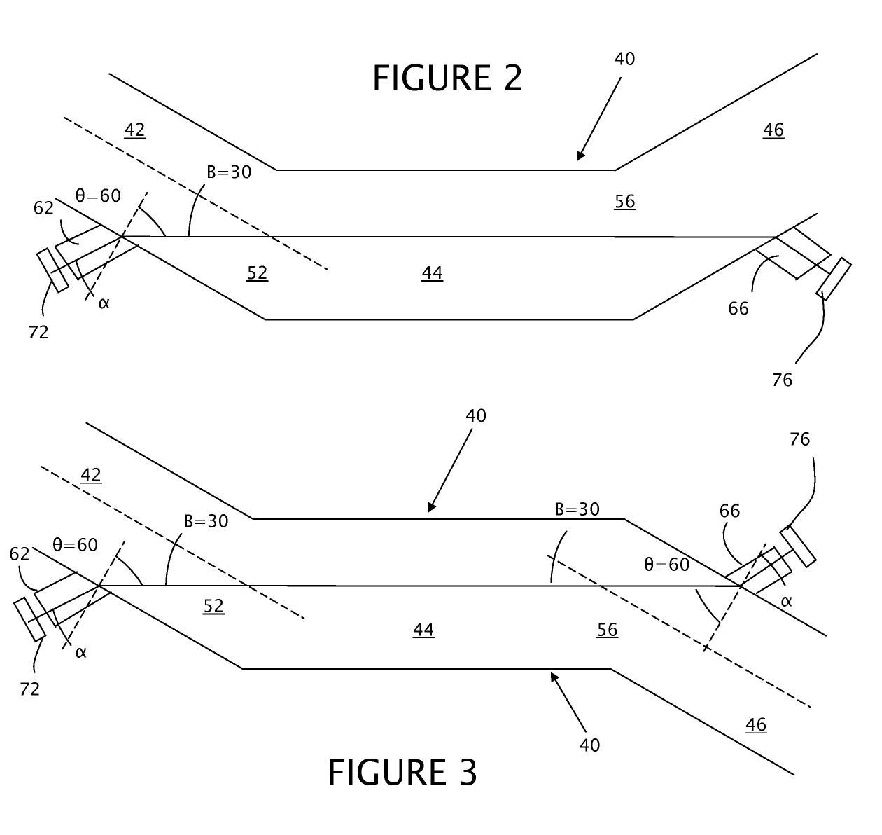

As seen in FIGS. 2, 3 and 12, in the flow sensor 22, the housing 30 defines a flow channel 40, wherein the flow channel includes an inlet channel 42, a linear section or measurement channel 44 and an outlet channel 46, wherein the inlet channel and the outlet channel interest the measurement channel at an angle B, as seen in FIGS. 2 and 3. That is, the inlet channel 42 and the outlet channel 46 are non collinear with the measurement channel 44. The measurement channel 44 has longitudinal axis extending along the length of the channel. The intersection of the inlet channel and the measurement channel occurs at a first or inlet bend 52 and the intersection of the outlet channel and the measurement channel occurs at a second or outlet bend 56.

The angle of intersection between the measuring channel 44 and the inlet and outlet channels 42, 46 is selected to provide relatively smooth passage of the flow into and out of the measuring channel. That is, the flow channel 40 defines a flow path through the housing 30 that avoids abrupt angles or corners which can otherwise introduce a fluidic “dead space” which can: (i) trap air bubbles that block ultrasound transmission; (ii) gradually accumulate proteins and other organic debris and possibly obstruct shunt flow and (iii) create flow turbulence and vortices at high flow rates, all of which may reduce flow rate measurement accuracy. The angle of intersection of the inlet and outlet channels 42, 46 with the measurement channel 44 is selected to reduce flow disturbance and debris accumulation along the measurement channel 44.

Referring to FIGS. 2, 3 and 12, in view of the anticipated conduit or shunt size into which the present flow sensor 22 can be spliced and the anticipated flow rate, a satisfactory intersection angle between the inlet channel 42 and the measurement channel 44 has been found to be approximately 30°. Similarly, a satisfactory exit angle from the measurement channel 44 to the outlet channel 46 has been found to be approximately 30°.

As seen in FIGS. 2, 4, 5 and 12, the linear length of the measurement channel 44 is selected to provide the desired or required measurement sensitivity. In one of the embodiments, the flow sensor 22 had a measurement channel 44 having a length between approximately 0.5 mm and 20 mm, with a satisfactory length of approximately 14 mm. In connection with the additional features of the flow sensor 22, the length of the measurement channel 44 within the implantable housing 30 can provide for measurement of flow rates less than 0.6 ml/hr.

Referring to FIGS. 2,3 and 4-8, the housing 30 defines a first signal pathway 62 adjacent the first bend 52 and a second signal pathway 66 adjacent the second bend 56. The first and second signal pathways 62, 66 are defined by the material of the housing 30 and terminate at one end at the flow channel 40, thereby defining a portion of the flow channel, and terminate at a free end spaced apart from the flow channel. The signal pathways 62, 66 are configured to conduct signals into the flow channel 40 and define a portion of the flow channel. In one configuration, the flow channel 40 is entirely defined by the material of the housing. That is, there is no separate or independent conduit or tube guiding flow through the housing 30, although it is contemplated that a conduit or tubing could be disposed within the housing to direct the flow. For example such tubing could be a portion of the CSF drainage shunt catheter itself.

As seen in FIG. 2, the geometry of the inlet channel 42, the measurement channel 44 and the outlet channel 46 locates the inlet channel and the outlet channel coplanar with the measurement channel, wherein the inlet channel and the outlet channel extend to form a generally concave, or D-shaped path. Alternatively, as seen in FIGS. 3-8, the geometry of the inlet channel 42, the measurement channel 44 and the outlet channel 46 locates the inlet channel and the outlet channel coplanar with the measurement channel, wherein the inlet channel and the outlet channel extend from different sides of the measurement channel to form a generally S or Z-shape.

The implantable housing 30 is sized for operation in a subcutaneous location. Typical dimensions for the implantable housing 30 are:

| Implantable housing dimensions | Infant | Adolescent child or adult |
|---|---|---|
| Width | ≦11 mm | ≦11 mm |
| Length | ≦20 mm | ≦25 mm |
| Thickness | ≦4 mm | ≦8 mm |

It is also contemplated that the implantable housing 30 can have an exposed surface that is shaped, contoured, or molded for cooperatively engaging or retaining the internal coupling coils.

The implantable housing 30 is formed of a biocompatible inert material, such as polyetherimide thermoplastic resins including those manufactured and sold by SABIC Innovative Plastics under the mark ULTEM®, as well as medical grade polysulfone, PEEK and silicones. It is also contemplated the housing 30 can be formed of injection-molded or machined medical ceramics or injection-molded or machined metals such as titanium or stainless steel 316L.

The ULTEM polyetherimide thermoplastic resins provide satisfactory housings 30, which can be machined to the desired configuration. The ULTEM material exhibits compatible acoustic properties, mechanical strength, and biocompatibility. The implantable housing 30 is generally formed of a single material, however, it is contemplated that multiple materials can be employed to form the housing.

The implantable housing 30 can include a resilient or compliant covering, such as soft silicone and specifically composed of an approximately 1 mm thick shunt grade silicone. The covering is selected to reduce pressure induced tissue breakdown resulting from contact with the implantable housing 30. As set forth below, the compliant covering may encapsulate the housing 30, which retains the internal coupling coils 80. Alternatively, the covering can encapsulate the housing 30 and the internal coupling coils 80 as the internal coupling coils are wrapped about at least a portion of the housing.

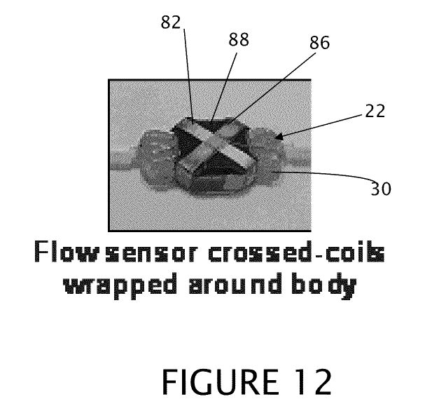

As seen in FIG. 12, although the present configuration is set forth in terms of an ultrasonic transit time flow sensor 22 in the housing 30, it is understood that any of a variety of sensors, detectors or devices can be employed within the housing or electrically connected to the housing such as, but not limited to a diagnostic sensor, an optical sensor, a thermal sensor, a pressure sensor, an electric sensor, or a chemical sensor.

Referring to FIGS. 1, 5, 10 and 12, the transit time ultrasonic flow sensor 22, operates under the principles as set forth in U.S. Pat. No. 4,227,407 to Drost and U.S. Pat. No. 7,194,919 to Shkarlet and Drost, both of which patents are hereby expressly incorporated herein by reference. For passing ultrasonic signals (waves) downstream through the flow, the transit time ultrasonic flow sensor 22 requires an upstream sender or emitter and a downstream receiver. For passing ultrasonic signals (waves) upstream through the flow, the transit time ultrasonic flow sensor requires a downstream sender or emitter and an upstream receiver. While the flow sensor 22 typically employs two ultrasonic transducers that alternately operate as emitter and receiver, it may be constructed using more piezoelectric transducers, wherein for instance two transducers are located at the upstream location and two transducers are at the downstream location. Therefore, in one configuration, the transit time ultrasonic flow sensor 22 has a single upstream transducer and a single downstream transducer, each transducer functioning as a transceiver as is well known in the art. For purposes of description, the transducers are referred to as a first transducer 72 and a second transducer 76. In terms of flow through the flow channel 40, one of the first and the second transducers 72, 76 can be an upstream transducer and a remaining one of the first and the second transducers can be a downstream transducer.

Referring to FIGS. 2 and 3, to accommodate for the angle of the inlet channel 42 and the outlet channel 46 relative to the measurement channel 44, the ultrasonic waves (signal) must refract upon passage from the signal pathway into the flow channel 40 so as to pass along, or parallel to the axis of the measurement channel. Referring again to FIGS. 2 and 3, the selected 30° intersection angle B of the inlet and outlet channels 42, 46 with the measurement channel 44 requires the ultrasonic waves refract by 60° to then pass along the length of the measurement channel 44, without reflecting off the walls of the flow channel 40, which can create phase errors that distort resulting flow measurements.

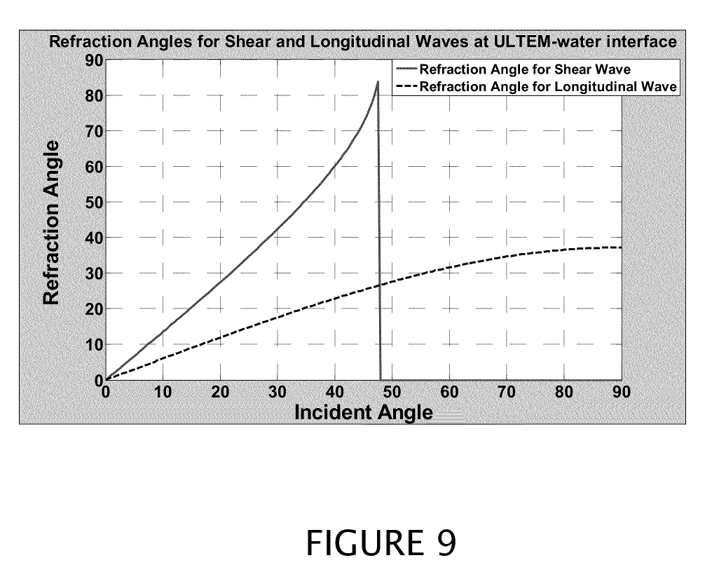

From Snell's law and for the data for the materials as shown in FIG. 9 and FIGS. 2 and 3, an ultrasonic shear wave incident angle at 40° to the inlet or outlet channel will refract at the required 60° angle. An advantage of refracting a shear wave is that a longitudinal wave will only refract at a maximum angle of 38°, much less than the required 60° for transmission parallel to the axis of the measurement channel 44. As seen from FIG. 9, the angle of incidence of an ultrasonic longitudinal wave and the resulting refraction precludes the use of a longitudinal wave passing from the signal pathway into the measurement channel 44 and then traversing along the measurement channel at least substantially parallel to the channel wall. Therefore, in this configuration the transducers 72, 76 include shear wave piezoelectric crystals, as are well known in the art.

| | | Longitudinal | Shear | | Longitudinal. | Shear |
|---|---|---|---|---|---|---|
| | | Wave | Wave | Absorption | Acoustic | Acoustic |
| | Density | Velocity | Velocity | at 4.8 MHz | Impedance | Impedance |
| Material | (gm/cm³) | (m/sec) | (m/sec) | (dB/cm) | 10⁶ kg/m³ | 10⁶ kg/m³ |
| ULTEM plastic | 1.3 | 2450 | 1100 | 0.54 | 3.2 | 1.4 |
| Polysulfone | 1.25 | 2280 | 1026 | 0.84 | 2.9 | 1.3 |
| Silicone (30A | 1.05 | 1000 | N/A | 2.0 | 1.0 | N/A |
| Hardness) | | | | | | |
| Water or CSF | 1.0 | 1527 | N/A | 0 | 1.5 | N/A |
| at 36° C. | | | | | | |

Referring to FIGS. 2 and 3, as shear waves cannot propagate in fluids, the shear wave generated by the respective transducers 72, 76 refracts at the solid-fluid interface between the signal pathway and the flow in the flow channel 40 and undergoes a mode conversion to a longitudinal wave as the wave enters the flow in the flow channel. After passing through the measurement channel 44, the longitudinal wave intersects the opposing signal pathway defining a portion of the wall of the flow channel 40 or in one configuration the measurement channel 44, the longitudinal wave refracts at the fluid-solid interface and converts back to a shear wave in the signal pathway. The converted shear wave then traverses the remaining signal pathway and is sensed by the remaining transducer.

Referring to FIGS. 2 and 3, the first or upstream transducer 72 is retained within the housing adjacent the first signal pathway 62. The first transducer 72 can be directly connected or attached to the first signal pathway 62. Alternatively, the first transducer 72 can be acoustically coupled to the first signal pathway 62. The second or downstream transducer 76 is retained within the housing adjacent the second signal pathway 66. The second transducer 76 can be directly connected or attached to the second signal pathway 66. Alternatively, the second transducer 76 can be acoustically coupled to the second signal pathway 66.

As seen in FIGS. 2 and 3, the connection of the respective transducers 72, 76 to the corresponding signal pathways 62, 66 and the angle of the signal pathway relative to the measurement channel 44 are selected such that the first transducer 72 produces a shear wave to pass along the first signal pathway 62, wherein the shear wave refracts and converts to a longitudinal wave at the interface of the first signal pathway and a flow in the flow channel 44. The angle of refraction is selected such that the resulting longitudinal wave propagates along the measurement channel 44, and refracts into the second signal pathway 66 to form a refracted shear wave in the second signal pathway 66.

The second transducer 76 is located adjacent the second signal pathway 66 to intersect the refracted shear wave passing though the second signal pathway.

Referring to FIGS. 1 and 12, thus, a wave mode converting transit time ultrasonic flow sensor 22 is provided. It is contemplated the wave mode converting transit time ultrasonic flow sensor 22 can be configured for monitoring CSF shunt flow in extra-ventricular drains (EVDs); as a diagnostic sensor for acute diagnostic use at any point along or on the distal end of a CSF shunt implant; or as a long-term implantable sensor integral to the CSF shunt implant for implantation near or on the skull. That is, the flow sensor 22 can be configured for use in the implantable housing 20 for subcutaneous operation, or directly coupled to a flow meter and power source in an extracorporeal application.

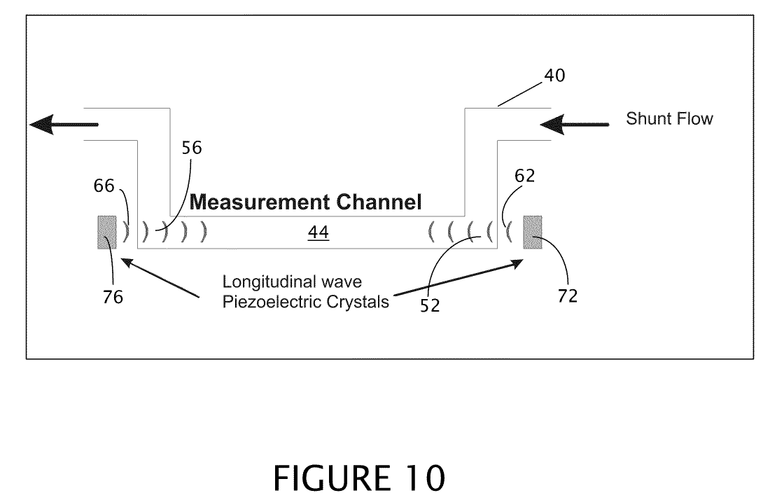

In an alternative configuration, the measurement channel can define a substantially U-shape, as seen in FIG. 10. In this configuration, the signal pathways 62, 66 are perpendicular to the intersected region of the flow channel 40. Therefore, there is no bending or refraction of the signal as the signal passes from the transducers 72, 76, through the associated signal pathways 62, 66 and into the measurement channel 44 parallel to the longitudinal axis of the measurement channel. That is, in the U-channel configuration of FIG. 10, both the first or upstream transducer 72 and the second or downstream transducer 76 are located in order to pass a longitudinal wave through an adjacent portion of the wall of the measurement channel 44 along an axis that is substantially perpendicular to the region of the channel wall through which the signal passes, and wherein the signal path (refracted signal path) is parallel to the longitudinal axis of the measurement channel 44. Similarly, the longitudinal wave traversing the measurement channel 44 then intersects the remaining signal pathway defining an internal surface of the measurement channel to pass from the measurement channel wall as a longitudinal wave. Thus, longitudinal wave producing transducers can be employed in this configuration of the flow sensor 22, shown in FIG. 12.

As seen in FIGS. 1, 11 and 12, in the implantable configuration, the implantable sensor 20 includes the internal coupling coils 80 which cooperate with the external coupling coils 90 which are retained outside of the patient. The internal coils 80 are selected to provide inductive coupling to the external coils 90, thereby establishing transcutaneous energy transfer. The internal coils 80 and the external coils 90 are windings of an elongate conductor such as metal. For purposes of description, the respective coupling coils 80 and 90 may be referred to as internal and external coils. Further, as previously set forth in one configuration of the flow sensor 22, a first transducer 72 and a second transducer 76 are employed. Thus, in this configuration the implantable sensor 20 includes a corresponding first and second internal coil 82, 86 and a first and second external coil 92, 96.

The external coils 92, 96 are disposed within or about a casing, wherein the casing is electrically connected to the flowmeter 100 by a cable or wire.

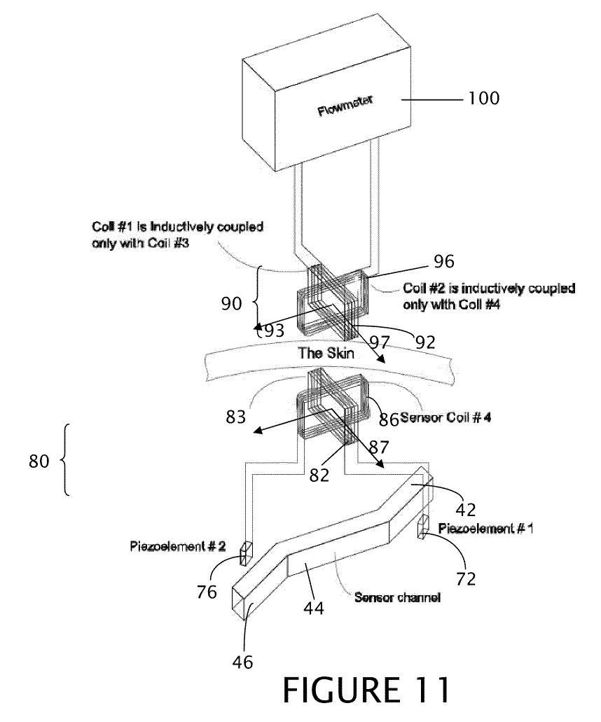

As seen in FIGS. 11 and 12, the internal coils 82, 86 can be retained entirely within the housing 30, or can be partially retained within the housing. In a further embodiment, the internal coils 82, 86 can be wrapped about at least a portion of the housing 30, wherein the internal coils are subsequently encapsulated by the covering with the housing 30, thereby forming a single encapsulated structure.

As seen in FIG. 11, the first internal coil 82 includes a first coil axis 83 and the second internal coil 86 includes a second coil axis 87. The first coil axis 83 is the axis about which windings of the first coil 82 are concentric and orthogonal to the plane in which the windings are disposed, and the second coil axis 87 is the axis about which windings of the second coil 86 are concentric and orthogonal to the plane in which the windings are disposed. Generally the windings within a given coil 82, 86 are coplanar, and for purposes of size reduction are wound to minimize the coil dimension along the respective coil axis.

In the configuration of FIGS. 1 and 11, the internal coils 82, 86 and the external coils 92, 96 cooperate to transcutaneously receive and send pulses from the flowmeter through the skin thickness to the transducers 72, 76 in the flow sensor 22.

As seen in FIG. 11, it is understood that one of the first and the second internal coils 82, 86 is an upstream coil as it is operably connected to the first or upstream transducer 72 and a remaining one of the first and the second internal coils 82, 86 is designated as a downstream coil as the coil is operably connected to the second or downstream transducer 76. In one configuration, the upstream internal coil 82 is directly electrically connected to the upstream transducer 72 and the downstream internal coil 86 is directly connected to the downstream transducer 76. By directly connecting each of the coils 82, 86 to a respective one of the transducers 72, 76, the costs and potential complications of additional circuitry and components in the implantable housing 30 are reduced.

Further, it is contemplated that the first internal coil 82 can be connected to the sensor 22 or a component of the sensor and the second internal coil 86 can be connected to a second sensor or a second component of the sensor.

In one configuration the internal coils 80 are crossed coils in that the coils 82, 86 are tilted or rotated relative to each other, and in one configuration the first internal coil 82 is perpendicular to the second internal coil 86. In the 90° tilted orientation, the first internal coil 82 and the second internal coil 86 and their associated magnetic fields are perpendicular to each other. In one configuration, the first coil axis 83 is perpendicular to the second coil axis 87, and the axes intersect.

Similarly, as seen in FIG. 11 the external coils 90 are crossed coils in that the coils 92, 96 are tilted 90° from each other, and in one configuration the first external coil 92 is orthogonal to the second external coil 96. In the 90° tilted orientation, the first external coil 92 and the second external coil 96 and their associated magnetic fields are perpendicular to each other.

While the description of the coil axes being tilted or rotated to a perpendicular orientation or alignment appropriately sets forth the relationship for symmetrical coils as shown in FIG. 11, it is understood the external coils 90 and internal coils 80 can have any of a variety of configurations, wherein the windings are not symmetric about or define a single coil axis. In these configurations, the coils 92, 96 are aligned to provide for substantially independent operation of the coil 92 with respect to the coil 96, and substantially independent operation of the coil 96 with respect to the coil 92. That is, the coils 92, 96 are oriented to provide substantially zero mutual inductance or zero coupling effect. Similarly the internal coils 82, 86 are aligned to provide for substantially independent operation of the coil 82 with respect to the coil 86, and substantially independent operation of the coil 86 with respect to the coil 82. That is, the coils 82, 86 are aligned to provide substantially zero mutual inductance or zero coupling effect between the internal coils. Thus, the first internal coil 82 can be oriented in a zero (or minimum) mutual inductance or zero (or minimum) coupling effect with respect to the second internal coil 86 (as well as the second external coil 96) while providing operable communication with the first external coil 92. Similarly, the second internal coil 86 can be oriented in a zero (or minimum) mutual inductance or zero (or minimum) coupling effect with respect to the first internal coil 82 (as well as the first external coil 92) while providing operable communication with the second external coil 96. The first external coil 92 can be oriented in a zero (or minimum) mutual inductance or zero (or minimum) coupling effect with respect to the second external coil 96 (as well as the second internal coil 86) while providing operable communication with the first internal coil 82. The second external coil 96 can be oriented in a zero (or minimum) mutual inductance or zero (or minimum) coupling effect with respect to the first external coil 92 (as well as the first internal coil 82), while providing operable communication with the second internal coil 96.

A zero mutual inductance or zero coupling effect is understood to encompass a non zero inductance or non zero coupling below a detrimental threshold. That is, if there is any inductive coupling between the two external coils 92, 96, the induced voltage is insufficient to create a detrimental effect on signal or power transmission and thus the relationship of the coils is effectively zero mutual inductance or zero coupling effect. Similarly, if there is inductive coupling between the internal coils 82, 86, the resulting voltage is insufficient to create a detrimental effect on signal or power transmission and impair the intended operating parameters of the system. Thus, the first internal coil 82 is disposed in a zero mutual inductance or zero coupling effect orientation with respect to the second internal coil 86. Similarly, the first external coil 92 is disposed in a zero mutual inductance or zero coupling effect orientation with respect to the second external coil 96.

Typically, the relative configuration and orientation of the internal coils 80 will be the same as the relative configuration and orientation of the external coils 90. Therefore, upon inductively coupling the external coils 90 to the internal coils 80, the coils 82, 92 provide sufficient mutual inductance to operably transfer the signal or power, without inducing a detrimental voltage in the coils 86, 96. Similarly, the coils 86, 96 provide sufficient mutual inductance to operably transfer the signal or power, without inducing a detrimental voltage in the coils 82, 92.

It is contemplated that the first internal coil 82 and the second internal coil 86 are nested such that an occupied volume of the coils is minimized, as seen in FIG. 11. In one construction, one of the first and the second internal coil 82, 86 is wound about a remaining one of the first and the second internal coil. That is, a portion of the windings of one of the coils encompasses a portion of the windings of a remaining one of the coils. However, it is understood the first internal coil 82 and the second internal coil 86 can be spaced apart within the housing 30, wherein the respective coil axes 83, 87 are perpendicular, and can be selected to be mutually orthogonal, as well as intersecting. With respect to the internal coils 80, the external coils 90 have the same relative orientation between the coil axes as dictated by the internal coils. Thus, the orientation between the coil axes in the internal coils 80 is the same as the orientation between the coil axes in the external coils 90.

As seen in FIG. 12, it least one, and typically both the first internal coil 82 and the second internal coil 86 can be wrapped around a ferrite core 88, wrapped about a ferrite disk on the exterior of the implantable housing 30 as seen or wrapped around the implantable housing as shown in FIG. 12.

Thus, the internal coils 82, 86 can be formed with or without cores 88 inside the respective coil. A satisfactory material for the core 88 is ferrite, such as a ferromagnetic oxide, which increases magnetic field strength.

As seen in FIG. 11, it is further contemplated different combinations of coils and cores 88 for the internal coils 80 and external coils 90 can be employed. For example, the external coils 90 can include the ferrite cores 88 and the internal coils 80 can be formed without the ferrite cores. Conversely, the external coils 90 can be formed without the ferrite cores and the internal coupling coils 80 can be formed with the ferrite cores 88. Alternatively, both the external coils 90 and the internal coils 80 can include the cores 88, or both the external coils and the internal coils can be formed without the ferrite cores. The core 88 is employed to increase the field strength and hence strength of transmitted signal, thereby improving the signal to noise ratio, without requiring larger diameter coils, which might act to increase susceptibility to ambient electrical noise. Thus, it is believed advantageous to include the ferrite cores 88 with either of the external coils 90 and the internal coils 80. To reduce the volume of the implantable housing 30, one configuration includes cores 88 in the external coils 90, wherein the internal coils 80 are free of the cores.

In operation, the alignment of the external coupling coils 90 and the internal coupling coils 80, the axes 83, 93 of the first coils 82, 92 respectively are aligned, so that the axes are parallel. Also, the axes 87, 97 of the second coils 86, 96 respectively are aligned, so that the axes are parallel. In one configuration, the first coil axis 83 is orthogonal to the second coil axis 87, such that the axes intersect and the coil axis 93 is orthogonal to the coil axis 97, such that the axes intersect.

The crossed coil construction provides a transcutaneous transmission distance of (13 mm with the ferrite core, and 9 mm without the ferrite core 88); acceptable flow measurement accuracy when coupled with wave converting sensor 22 configuration; and relative ease at with which the external coils 90 are aligned with the internal coils 80.

Representative design parameters include:

| | External coil | | Internal coil | | Internal coil | |
|---|---|---|---|---|---|---|
| | with ferrite | | with ferrite | | without ferrite | |
| Feature | core | | core | | core | |
| Coil width | 4.75 | mm | 2.5 | mm | 2.5 | mm |
| number of wire turns | | wide, | | wide, | | wide, |
| | | 30 turns | | 20 turns | | 20 turns |
| Long Axis Diameter | 12.5 | mm | 12.5 | mm | 15 | mm |
| Short Axis Diameter | 0.75 | mm | 0.75 | mm | 4.5 | mm |
| Maximum skin | 9-13 | mm | 9 | mm | 13 | mm |
| thickness for | | | | | | |
| transmission | | | | | | |

As shown in FIGS. 11 and 12, in the flow sensor 22, it is further contemplated that the operating frequency of the transducers 72, 76 can be approximately 9.6 MHz. The approximately 9.6 MHz operating frequency will provide the flow sensor 22 with sustained accuracy for a shorter length of the measurement channel 44, which in turn will allow the overall length of the housing 30 to be shorted and thus compatible with pediatric size requirements. However, alternative frequencies can be employed such as, but not limited to 4.8 Mhz.

A satisfactory flowmeter 100 has been found to be a modified standard TS-420 Ultrasonic Transit-Time flowmeter by Transonic Systems Inc. of Ithaca N.Y. The modified flowmeter 100 is configured to operate the inductively coupled external and internal coils 80, 90 and determine a flow from the implanted flow sensor 22, via the signals from the transducers 72, 76.

The modifications to the TS-420 Ultrasonic Transit-Time flowmeter 100 include an increased voltage meter transmit signal output stage which energizes the piezoelectric transducers of the implanted flow sensor. The modified flowmeter 100 provides a larger transducer transmit voltage amplitude and longer transmit signals to take advantage of the increased flow measurement resolution features of the implanted flow sensor 22. Also, the flowmeter 100 is modified to provide a visual display of the signal coupling between the external flowmeter and the implanted flowsensor 22, so as to allow optimal alignment between the external and internal coil axes.

These modifications allow the TS-420 Ultrasonic Transit-Time flowmeter 100 to operate the inductively-coupled shunt flow sensor 22 over a distance of 9 mm for non-ferrite coupling coils 80, 90, and up to 13 mm with ferrite core coupling coils 80, 90.

Referring to FIG. 11, the present flowmeter 100 can also include a zero offset for accommodating relative motion between in the coupling coils 80, 90 in the transcutaneous energy transfer. That is, relatively large relative motion between the external (extracorporeal readout) coupling coils 90 and the internal (implanted) coupling coils 80 strongly alter the received signal strength and temporarily change the zero offset (zero offset is the meter reading under zero flow conditions) of the signal.

The amplitude of the signal received at the external coils 92, 96 is inversely proportional to the coil-to-coil separation distance between the internal coils 80 and the external coils 90. It has been found that a change in the zero offset can be corrected for by monitoring the signal amplitude received at the external coupling coils 90.

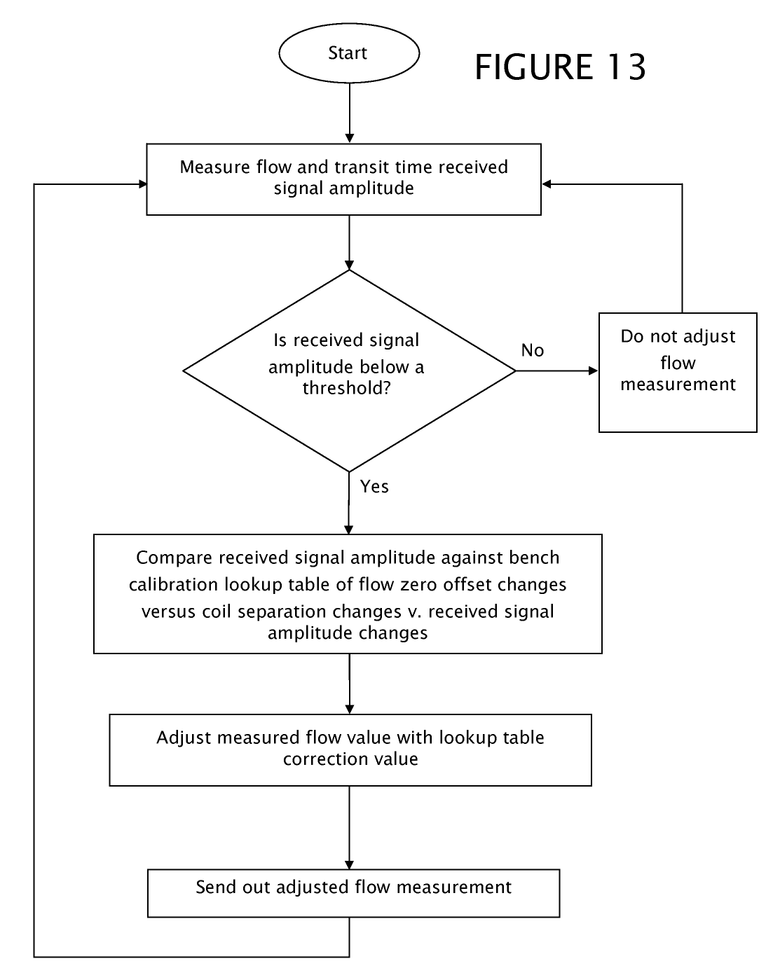

Referring to FIG. 13, an algorithm is provided for accommodating zero offset changes. Generally, the received signal amplitude is compared to a predetermined threshold. For those received signals having an amplitude above the threshold, the received signal is processed without a correction factor. For those received signals having an amplitude below the predetermined threshold, the amplitude of the received signal is compared to a calibration table, such as may be previously determined from bench testing, wherein the calibration or lookup table correlates amplitude changes of the received to coil separation, and a zero offset change. The measured amplitude of the received signal below the predetermined threshold is then adjusted in accordance with the lookup table to provide a corrected value, and the corrected value is then processed by the flowmeter 100 to provide an adjusted flow reading. It is contemplated that the correction data can be expressed as the lookup table or a calibration table or a mathematical function or relation derived from the correction data.

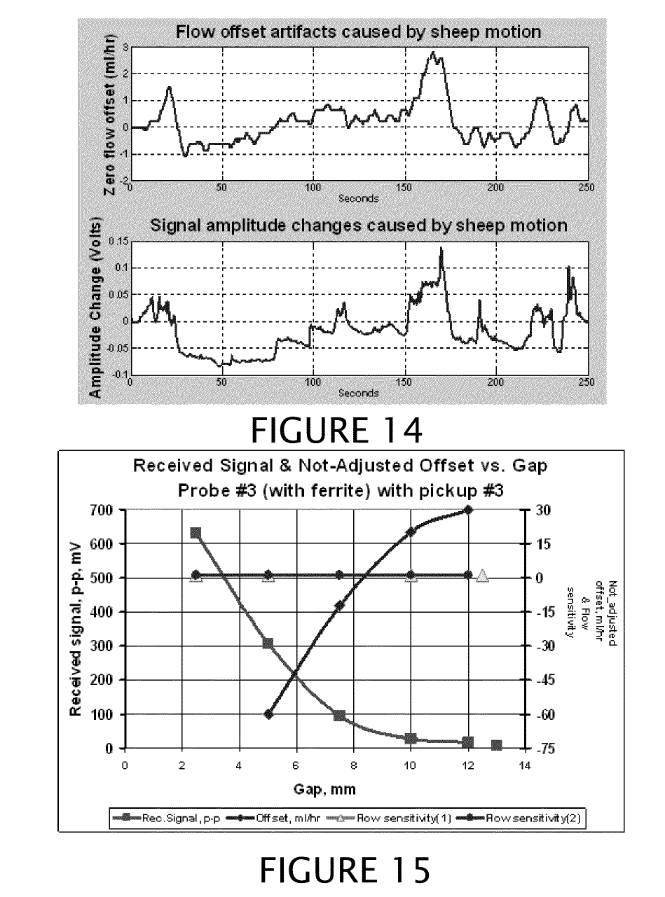

FIG. 14 shows data from an in-vivo study in a sheep, in which flow was stopped in order to assess the effects of sheep motion on flow zero offset. The plot show how changes in zero offset track changes in the received signal voltage amplitude. That is, the changes in coil alignment and/or separation correlate to flow zero offset correction and such changes can be used to provide zero flow offset correction.

As seen in the Table below, the correction algorithm compensates the recorded flow or motion induced changes to zero flow offset.

| Pump | Flowmeter | | | |
|---|---|---|---|---|
| Flow | Reading | % Absolute | Flowmeter Reading | % Absolute |
| (ml/hr) | (ml/hr) | Error | Compensated (ml/hr) | Error |
| 0 | 1.07 | | 0.00 | |
| 120 | 133 | 10.8 | 133.30 | 10.8 |
| 80 | 86.9 | 8.6 | 88.03 | 10 |
| 60 | 63.6 | 6.0 | 65.63 | 9.3 |
| 30 | 27.8 | −7.3 | 31.90 | 6.3 |
| 10 | 3.54 | −64.6 | 10.19 | 1.9 |
| 6 | −0.43 | −107 | 6.25 | 4.2 |
| 3 | −2.68 | −189 | 3.22 | 7.3 |

Thus, the flowmeter 100 can be programmed or configured to continuously monitor changes to the amplitude of the received signal. By monitoring changes to the amplitude of the received signal, the flowmeter 100 can correct the flow signal offset using lookup tables calculated from bench calibration data or alert the operator to perform a new zero calibration. For implanted sensors monitoring CSF flow, density measurements could also be used as adjunct measurements to correlate CSF density changes with shunt function. For example, density changes could be caused by infections. Similarly, for extracorporeal sensors, and when used to monitor cranial drainage from patients recovering from brain surgery, density changes could give early warning of bleeding or infection by monitoring the fluid density changes.

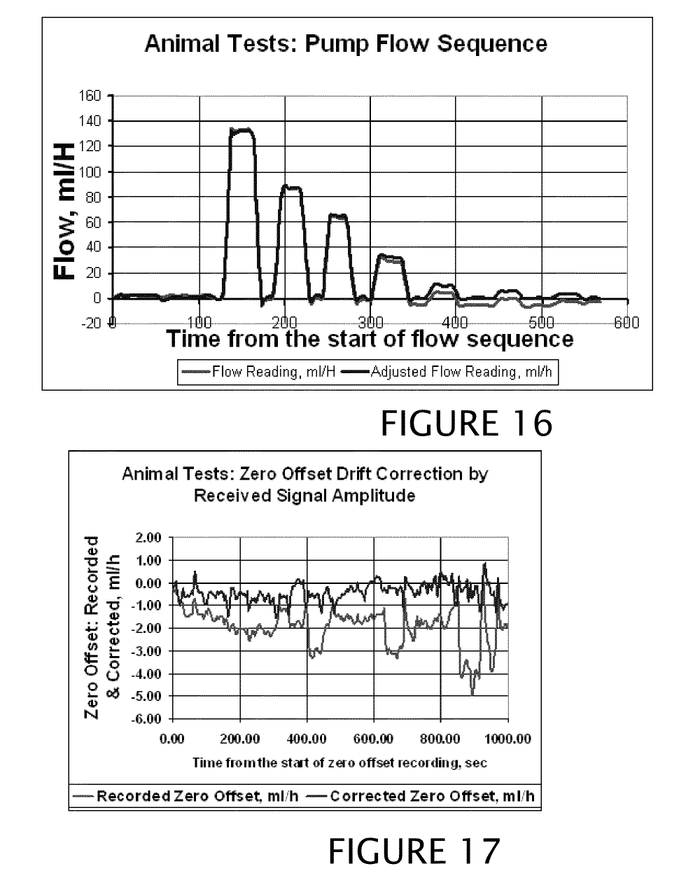

Referring to FIG. 16, the correlation of actual flow to a zero offset compensated measure of flow is shown. In FIG. 17, the zero offset drift correction by the flow meter 100 based on the amplitude of the received signal is shown. Therefore, the zero offset compensation can be used to compensate for relative motion between the external coils 90 and the internal coils 80.

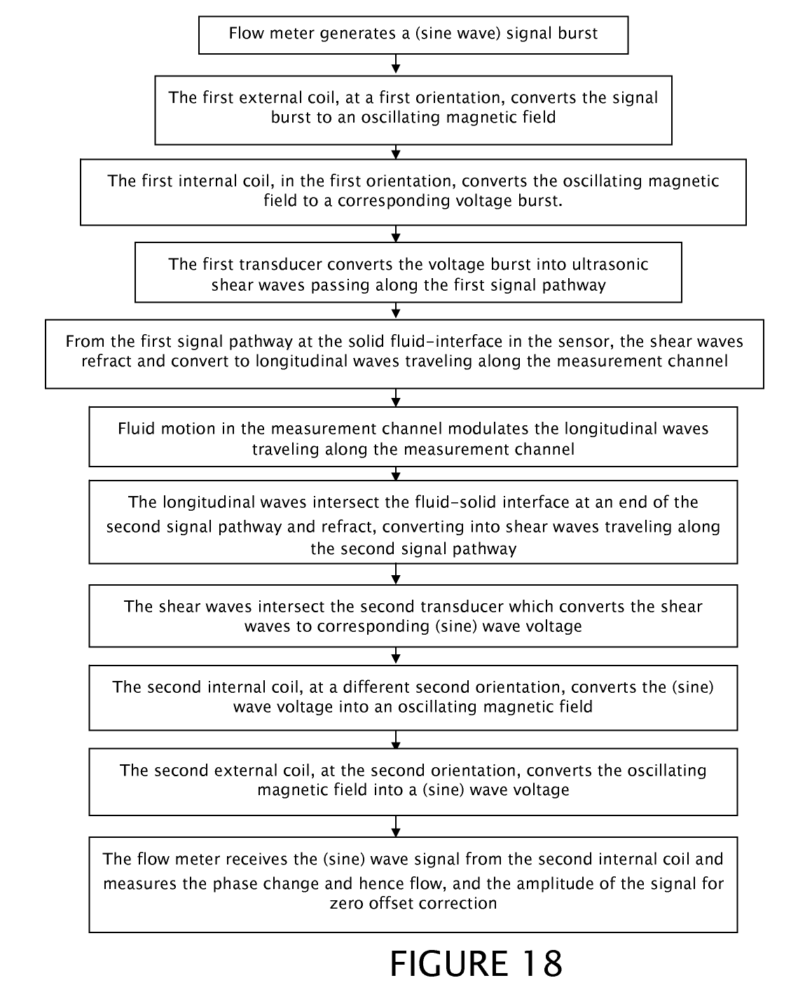

Referring to FIG. 18, the operation of the implantable flow sensor 22 and transcutaneous energy and signal transfer is provided. While the operation is set forth in terms of the signals and voltages in the form of sine waves, it is understood that any signal pattern can be employed.

The flowmeter 100 generates a (sine wave) signal burst or pulse, such as a sine wave. The sine wave signal burst travels through wires to the external coupling coils 90, and specifically through the first external coil 92. The first external coil 92, with the coil axis 93 at a first orientation, converts the signal burst to an oscillating magnetic field emanating from the coil.

The first internal coil 82, with the coil axis 83 in the first orientation, converts the oscillating magnetic field to a corresponding current and voltage burst. As the second internal coil 86 is oriented in a second (orthogonal) orientation to the first internal coil 82, there is no induced current or voltage in the second internal coil.

The induced voltage burst in the first internal coil 82 travels through wiring directly to the first transducer 72. The first transducer 72 converts the voltage burst into ultrasonic shear waves passing along the first signal pathway 62. The shear waves translate along the first signal pathway 62, and refract into the flow channel 40 to become longitudinal waves parallel to the measurement channel 44.

As the longitudinal waves travel in the flow in the measurement channel 44, the propagation speed of the waves is altered or modulated. The altered longitudinal waves intersect the second signal pathway 66 and refract into the pathway, converting into shear waves. These shear waves travel the second signal pathway 66 to intersect the second transducer 76. The second transducer converts the shear waves to corresponding sine wave voltage. The sine wave voltage travels through the wires directly to the second internal coil 86.

The second internal coil 86, with the coil axis 87 at the different second orientation, converts the sine wave voltage into an oscillating magnetic field emanating from the second internal coil. The second external coil 96, with the coil axis 97 at the second orientation, converts the oscillating magnetic field into a sine wave voltage or signal. As the axis 93 of the first external coil 92 is tilted or orthogonal to the axis 97 of the second external coil 96, a voltage is not induced in the first external coil from the magnetic field emanating from the second internal coil 86.

The sine wave signal from the second external coil 96 travels to the flowmeter 100. The flowmeter 100 receives the sine wave signal and measures the phase changes between this signal and a stationary reference signal, and the amplitude of the signal for zero offset correction.

A corresponding process is employed to send a longitudinal wave upstream from the second downstream transducer 76 to the first upstream transducer 72. That is, the process of FIG. 18 is reversed, so that the upstream and the downstream transit times of the ultrasonic signal are sequentially measured, and the difference is proportional to volumetric flow. It is further understood the order of the these processes can be alternated, reversed or performed simultaneous. Thus, the present system provides for the sequential or the simultaneous transcutaneous power transfer between, for example coupled coils 82 and 92 with transcutaneous signal transfer between, for example coupled coils 86 and 96.

It is further contemplated that the measurement of phase changes of the signals having passed through the measurement channel 44 can be employed in a number of distinct blood properties measurement approaches. The difference in phase measurement between two signals that have passed the measurement channel in the upstream and downstream direction, respectively, is proportional to flow through the implanted flow conduit such as set forth, for instance, in U.S. Pat. No. 4,227,407 to Drost, hereby expressly incorporated by reference. The average of the upstream and downstream signal (the “common mode phase signal”) is invariant to fluid flow but it may be monitored for changes in the time of flight of the ultrasound signal traveling from one transducer to the other in the implanted sensor. This time of flight is a function of the distance between these two transducers, and the ultrasound velocity of materials placed between these two transducers. The common mode phase signal will change to reflect changes in the acoustical velocity of the liquid in the measurement channel. Such changes in the acoustical velocity may result from temperature changes of the liquid, or from changes in the density or constituents of the liquid. By monitoring the phase changes of the signals traversing the measurement channel 44, the flowmeter 100 can determine temperature or density changes for purposes of measurements such as indicator dilution when a liquid parameter change such as a temperature change or a saline bolus injection is introduced into the flowing liquid, and as a measurement of hematocrit changes if the liquid flowing through the sensor were blood.

Referring to FIG. 10, in the U-shape flow channel 40, it is understood the above process can be employed, excepting that the transducers 72, 76 generate longitudinal waves.

A further aspect of the implantable sensor 20 is magnetic resonance imaging (MRI) compatibility.

In preliminary testing of the implantable sensor 22, artifact size caused by the presence of the sensor in the MRI and MRI induced force, torque, and RF (radio-frequency) heating were examined in a Philips Intera 1.5 Tesla whole body MRI scanner.

MRI induced force and torque were evaluated by hanging the sensor having the ferrite core and the sensor without the ferrite core from threads and moving the sensors into the 1.5 Tesla static field of the MRI machine and measuring the deflection angle. RF heating was evaluated by attaching the ferrite sensor to a liquid-filled phantom and conducting a T1 Weighted Spin Echo (1.5 Tesla, 63 MHz, 20-minute duration) scan and a GRE Gradient Recall Echo (20 minute duration) scan.

The MRI artifact was measured by suspending the non-ferrite sensor in a cylinder of water doped with an MRI contrast agent (Ominiscan diluted 1 part in 500), and repeating the above scans. The NIH Image-J image analysis package was used to analyze the scan DICOM image sequence and estimate the artifact dimensions.

The table below documents test results when prototype sensors were tested against four ASTM (American Society for Testing and Materials) standards for implanted medical devices exposed to a Magnetic Resonance Environment. Specifically, (i) ASTM F2052-06e1 Standard Test Method for Measurement of Magnetically Induced Displacement Force on Medical Devices in the Magnetic Resonance Environment; (ii) ASTM F2213-06 Standard Test Method for Measurement of Magnetically Induced Torque on Medical Devices in the Magnetic Resonance Environment; (iii) ASTM F2182-02a Standard Test Method for Measurement of Radio Frequency Induced Heating Near Passive Implants During Magnetic Resonance Imaging and (iv) ASTM F2119-01 Historical Standard: ASTM F2119-01 Standard Test Method for Evaluation of MR Image Artifacts from Passive Implants. This standard has been superseded by: ASTM F2119-07 Standard Test Method for Evaluation of MR Image Artifacts from Passive Implants.

| | | Torque | | |
|---|---|---|---|---|
| | Force | (preferred | RF Heating | |
| Sensor | (measured | axis | (Before/after | Larges MRI Artifact |
| Model | angle) | observed?) | temperatures) | Noted |
| With | 90° | Yes | Cool to touch | 5.6 cm × 2.6 cm × |
| ferrite | | | before/after | 1.8 cm (T1 |
| | | | scanning | Weighted) |
| | | | | 4.6 cm × 4.0 cm × |
| | | | | 2.9 cm (GRE) |
| Without | 0.5° | No | Cool to touch | Not measured |
| ferrite | | | before/after | because device did |
| | | | scanning | not pass Force |
| | | | | requirement. |
| ASTM | <45° | None | <3° rise | Must measure, but |
| Req. | | | | no requirements on |
| | | | | artifact size |
| ASTM | F2052- | F2213-06 | F2182-02a | F2119-01 |
| Document | 06e1 | | | |

The largest measured MRI artifacts were 5.6 cm×2.6 cm×1.8 cm (T1 Weighted), and 4.6 cm×4.0 cm×2.9 cm (GRE). These artifact sizes are comparable with those of programmable shunt valves.

In one configuration, the implanted sensor 22, with internal coils is compatible with following MRI machine specs: Static Magnetic Field: up to 1.5 Tesla; Gradient Magnetic Field: 14.91 Tesla/second vertical to patient, 26.17 Tesla/second along patient length, with a Radio-Frequency: 64 MHz (300-400 MHz in high-field head coils).

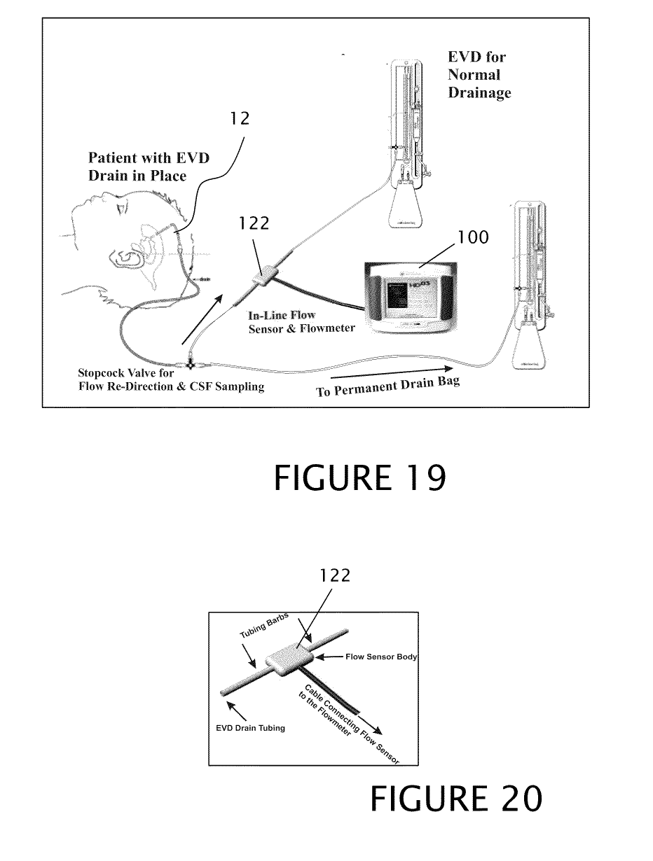

As seen in FIGS. 19 and 20, it is also contemplated that an extracorporeal version 122 of the wave mode converting transit time ultrasonic flow sensor can be employed for extracorporeal real time measurement of CSF flow of patients and for other flow and liquid properties measurements. That is, the extracorporeal flow sensor 122 can be directly connected to or hard wired to the flowmeter 100 for real-time measurement of flow through the sensor, such as CSF flow. Thus, the sensor 122 can be formed without the associated coupling coils, as power can be directly supplied to and signals received from the transducers.

For example, for patients connected to Extra-Ventricular Drains (EVDs), the flow sensor 122 can be spliced directly into a drainage line as shown in FIG. 19. The flow information from the flow sensor 122 in combination with the EVD drain bag elevation provides insight into the intracranial pressure versus flow pattern for the patient, and may result in a more informed shunt valve pressure setting selection.

Additionally, for patients with an implanted CSF shunt, there is a need for diagnosis of shunt functionality before the shunt is replaced. The flow sensor 122 can be spliced into existing exteriorized shunt tubing, and directly measure the flow. This again can allow the doctor to assess the functionality and flow-pressure relationship of the implanted CSF shunt in situ.

Referring to FIG. 20, the extracorporeal flow sensor 122 can be injection molded from polysulfone, medical-grade plastics, ceramics, or metals, rather than machined from ULTEM plastic. The body is approximately 17 mm long, 11 mm wide and 4 mm thick. However, in view of the extracorporeal location of this configuration of the flow sensor 122, the housing can be larger, thereby providing a longer measurement channel. The longer measurement channel is compatible with a lower frequency transducer, such as a 4.8 MHz shear wave piezoelectric crystals to generate the transit-time ultrasonic sine bursts. The 4.8 MHz frequency has historically been a good match for vessel diameters similar to the shunt drainage tubing, and this frequency is already supported by the Transonic TS-420 flowmeter.

In one configuration, the flow sensor 22, 122 can measure hydrocephalic shunt flows between approximately 0.5 ml/hr (one drop every 4 minutes) to 120 ml/hr. Further, the transcutaneous energy transfer is operable from approximately 5 mm to 10 mm skin (tissue) depth, and depending on the configuration of the crossed coils up to approximately 20 mm. In one configuration, a flow resolution of 0.5 ml/hr was obtained for a coil separation of 7.5 mm with the coils having core 88, and a coil separation of 6 mm for coils without the cores. At a coil separation distance of 13 mm for the coils having the core 88 and 9 mm for the coils without the core, the flow resolution was 1.0 ml/hr.

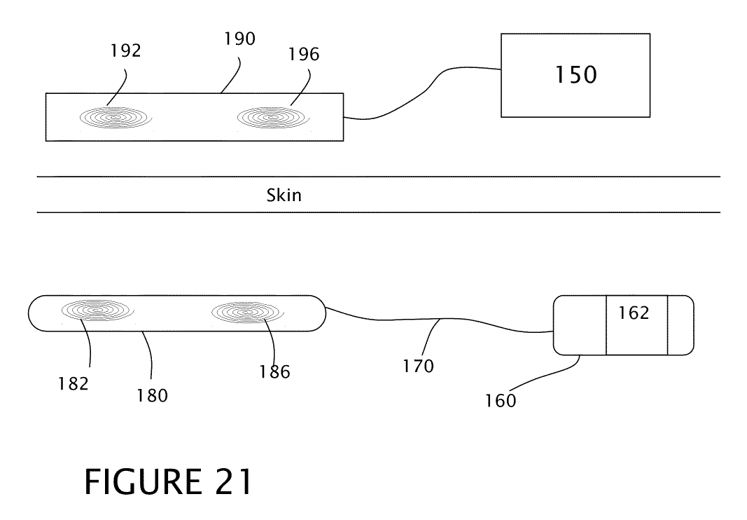

Referring to FIG. 21, a further configuration encompasses an implantable assembly, wherein the TET occurs remote from the implanted biological support device or sensor 162. In this configuration, an implantable first housing 180 retains at least one and typically two inductive coupling coils 182, 186 and an implantable second housing 160 retains the biological device or sensor 162 and an electrical conductor 170 connects the biological device or sensor to the implanted inductive coupling coils. Thus, the subcutaneous first housing 180 is electrically connected to the subcutaneous second housing 160 by a wire 170. The wire 170 is any of a variety of medical grade subcutaneous conductors and is typically encapsulated in a medical grade inert plastic such as disclosed above.

The biological support devices include pumps for introducing medicines, tracers or indicators, dispensers, heaters, coolers or even electrical stimulators. The implanted sensors include flow, pressure, ECG, EMG EEG, PH, and blood property sensors.

The external coupling coils 192, 196 are operably connected to a corresponding processor or control unit 150, as dictated by the corresponding implanted device 162.

This configuration provides for the operable location of the coupling coil(s) to be spaced from the subcutaneous device powered by the coupling coils. Therefore, detrimental effects associated with the inductive coupling can be spaced from the biologic support device. This separation is particularly advantageous when the biologic support device must be subcutaneously located proximal to a relatively sensitive or delicate area of the patient or wherein the size of the biologic support device must be minimized. As the coupling coils are remotely located subcutaneously, the biologic device can be configured to minimize size, as necessary power is supplied from the remotely located coupling coil.

In this configuration, the coupling coil can be a single coil or a plurality of coils in any of a variety of configurations. For example the coils 182,186,192,196 can be relatively planar structures concentrically would about a corresponding axis, wherein the coils 182, 186 are generally coplanar, and the coils 192, 196 are generally coplanar. It is also understood the internal and external coils can be those previously set forth as the internal coils 80 and the external coils 90, wherein the internal coils are not commonly housed or encapsulated with the implanted sensor.

Thus, the present disclosure provides transcutaneous signal and power coupling with two orthogonal coil pairs, one pair being subcutaneous and the other pair being external. Although described in terms of a hydrocephalus monitor application, the system has wide uses in any transcutaneous signal/power coupling for implanted (subcutaneous) sensors/controllers (passive such as the present ultrasonic sensor, and active meaning that the implanted device includes electronics that need to be electrically powered for the device to function or operate.) The subcutaneous coil set could be made part of the sensor housing 30, or connected via electronic cabling 170 to the sensor. The two subcutaneous coils may be fabricated co-axially or mounted side by side, wherein the requirement is met that the coils be positioned in relationship to each other such that their magnetic field coupling from one external/internal pair exhibits only minimal cross talk with the other external/internal pair. Thus, in addition to the present transcutaneous signal/power transfer application, the system can be employed in any non-contact measurement applications, such as in the measurement of parameters inside an isolation chamber, container, barrier or layer, where the spread of disease agents or chemically materials needs to be contained.

While a preferred embodiment of the invention has been shown and described with particularity, it will be appreciated that various changes in design and formulas and modifications may suggest themselves to one having ordinary skill in the art upon being apprised of the present invention. It is intended to encompass all such changes and modifications as fall within the scope and spirit of the appended claims.

1. An implantable sensor assembly comprising:
(a) an implantable housing;
(b) a first internal coil retained within the housing;
(c) a second internal coil retained within the housing, the second internal coil being in a zero mutual inductance orientation relative to the first internal coil; and
(d) a sensor connected to the housing and electrically coupled to at least one of the first internal coil and the second internal coil.

2. The implantable sensor of claim 1, wherein the sensor includes a first transducer and a second transducer, the first transducer electrically connected to the first internal coil and the second transducer electrically connected to the second internal coil.

3. The implantable sensor of claim 1, wherein the sensor is an ultrasonic flow sensor.

4. The implantable sensor of claim 3, wherein the ultrasonic flow sensor includes a flow channel having a linear section bounded by an inlet bend and an outlet bend.

5. The implantable sensor of claim 3, wherein the ultrasonic flow sensor includes a shear wave transducer.

6. The implantable sensor of claim 1, wherein the first internal coil has a first coil axis and the second internal coil has a second coil axis, the first coil axis being perpendicular to the second coil axis.

7. The implantable sensor of claim 1, wherein the first coil axis intersects the second coil axis.

8. The implantable sensor of claim 1, wherein at least one of the first internal coil, the second internal coil, the first external coil and the second external coil includes a high magnetic permeability core.

9. The implantable sensor of claim 1, wherein each of the first internal coil and the second internal coil includes a plurality of coplanar windings.

10. A method of transcutaneously transferring power, the method comprising:
(a) subcutaneously locating a first internal coil and a second internal coil, the first internal coil being oriented in a zero coupling orientation with respect to the second internal coil; and
(b) inductively coupling a first external coil with the first internal coil.

11. The method of claim 10, further comprising electrically powering an implantable sensor from at least one of the first internal coil and the second internal coil.

12. The method of claim 10, further comprising connecting one of the first internal coil and the second internal coil to a subcutaneous sensor.

13. The method of claim 10, further comprising aligning a first coil axis defined by the first internal coil perpendicular to a second axis defined by the second internal coil.

14. The method of claim 10, further comprising defining a first coil axis by the first internal coil and a second coil axis by the second internal coil and disposing a first external axis of a first external coil parallel to the first coil axis.

15. The method of claim 10, further comprising simultaneously passing a current though the first internal coil and the second internal coil.

16. A transcutaneous energy transfer assembly comprising:
(a) an external casing retaining a first external coil and a second external coil, the first external coil disposed in a zero coupling effect orientation relative to the second external coil; and
(b) an implantable housing retaining a first internal coil and a second internal coil, the first internal coil disposed in the zero coupling effect orientation relative to the second internal coil.

17. The assembly of claim 16, further comprising a sensor at least partially retained within the implantable housing, the sensor electrically connected to at least one of the first internal coil and the second internal coil.

18. The assembly of claim 16, wherein the a first internal coil has a first coil axis, the second internal coil has a second coil axis and the first coil axis is perpendicular to the second coil axis.

19. The assembly of claim 18, wherein the first internal coil is spaced from the second internal coil, and the first coil axis is perpendicular to the second coil axis.

20. The assembly of claim 16, wherein the first internal coil and the second internal coil are nested.

21. The assembly of claim 16, further comprising a subcutaneous device electrically connected to one of the first internal coil and the second internal coil.

22. The assembly of claim 16, wherein the implantable housing, the first internal coil and the second internal coil are compatible with magnetic resonance imaging.

23. A method of transcutaneously powering a subcutaneous device, the method comprising:
(a) subcutaneously locating a subcutaneous device, a first internal coil and a second internal coil, the first internal coil disposed in a zero mutual inductance orientation relative to the second internal coil;
(b) inductively coupling a first external coil with the first internal coil, and a second external coil with the second internal coil; and
(c) energizing the subcutaneous device from one of the first internal coil and the second internal coil.

24. The method of claim 23, further comprising aligning the first external coil parallel to the first internal coil.

25. The method of claim 23, further comprising employing one of a biological support device and a sensor as the subcutaneous device.

26. The method of claim 23, further comprising simultaneously passing a current though the first internal coil and the second internal coil.

27. A flow sensor comprising:
(a) a housing formed of a first material, the housing defining a flow channel having a linear section bounded by a first bend and a second bend, the first material of the housing forming a first signal pathway adjacent the first bend and a second signal pathway adjacent the second bend;
(c) a first transducer adjacent the first signal pathway; and
(d) a second transducer adjacent the second signal pathway.

28. The flow sensor of claim 27, wherein the first transducer is directly connected to the first signal pathway.

29. The flow sensor of claim 27, wherein the second transducer is directly connected to the second signal pathway.

30. The flow sensor of claim 27, wherein the first transducer is acoustically coupled to the first signal pathway.

31. The flow sensor of claim 27, wherein the second transducer is acoustically coupled to the second signal pathway.

32. The flow sensor of claim 27, wherein the first transducer produces a shear wave to pass along the first signal pathway, the first signal pathway selected to refract the shear wave into a fluid flow in the flow channel and convert the shear wave to a longitudinal wave to propagate along the linear section and refract into the second signal pathway to form a refracted shear wave in the second signal pathway.

33. The flow sensor of claim 27, wherein the second transducer is located to intersect the refracted shear wave passing though the second signal pathway.

34. The flow sensor of claim 27, wherein at least one of the first and the second transducer generates signals corresponding to at least one of a velocity of a fluid flow in the flow channel and a density of the fluid.

35. The flow sensor of claim 27, wherein at least one of the first and the second transducer generates signals corresponding to at least one of a velocity of a fluid flow in the flow channel and a temperature of the fluid.

36. An subcutaneous assembly comprising:
(a) a first subcutaneous housing;
(b) one of a sensor and a biological support device at least partially retained within the first subcutaneous housing;
(c) a second subcutaneous housing;
(d) at least one coupling coil at least partially retained with the second subcutaneous housing; and
(e) a subcutaneous conductor electrically connecting the biological device and the one coupling coil.

37. The subcutaneous assembly of claim 36, further comprising a second coupling coil in the first subcutaneous housing.

38. The subcutaneous assembly of claim 37, wherein the second coupling coil is in a zero mutual inductance orientation relative to the one coupling coil.

39. The subcutaneous assembly of claim 36, wherein the sensor is a flow sensor.

40. The subcutaneous assembly of claim 36, wherein the sensor is an ultrasonic transit time flow sensor.
